(19) United States
(12) Patent Application Publication
(10) Pub. No.: US 2012/0209280 A1
(43) Pub. Date: Aug. 16, 2012

(54) EYE MARKER FOR EYE SURGERY

(76) Inventor: Peter Leo Macken, Bowral (AU)

(21) Appl. No.: 13/500,923

(22) PCT Filed: Oct. 8, 2010
(86) PCT No.: PCT/AU10/01325

§ 371 Date: May 3, 2012

(30) Foreign Application Priority Data
Oct. 9, 2009 (AU) 2009904957

(51) Int. Cl. A61F 9/007

(52) U.S. Cl. 606/107

(57) ABSTRACT

An eye marking device, for marking an eye of a patient prior to eye surgery, said eye marking device comprising: a frame comprising a front face, a rear face and at least one marking tab extending from the front face; a removably attachable handle; and a handle connection means adapted to enable removable attachment of the handle to the frame in more than one orientation.

### FIELD OF THE INVENTION

The present invention relates to a surgical tool for eye surgery. In particular, the invention relates to a tool for assisting with the placement of reference marks on the cornea, prior to eye surgery, such as cataract surgery or refractive surgery.

### BACKGROUND OF THE INVENTION

Astigmatism commonly causes blurred vision for patients. It is usually due to an abnormality in the curvature of the cornea. This is referred to as corneal astigmatism. Corneal astigmatism is caused by differences in the radius of curvature in one principal meridian of the cornea compared to the other. These two principal meridians are located most commonly approximately at right angles to each other. One meridian is steeply curved (the steep axis) and the other meridian is less curved (the flat axis). To illustrate this difference, a soccer ball is spherical with equal radii of curvature. On the other hand a rugby ball has different radii of curvature. When light rays hit the steep meridian of an astigmatic cornea, they are refracted to a greater extent than the light rays which hit the flat meridian of the cornea. The light rays entering the cornea are then focused at two positions or planes inside the person's eye, instead of at a single point on the retina (the nerve-rich lining inside the eye which ‘reads’ the image received by the eye). Accordingly, the person's visual image is out of focus at the retinal plane. The result is that vision is generally blurred and this blurred vision can occur at short, intermediate and long viewing distances.

Patients with corneal astigmatism usually require spectacles to overcome astigmatism, correcting the refractive error, and providing clarity of vision. Sometimes astigmatism may be corrected with contact lenses. However, many patients prefer good vision without the need to wear spectacles or contact lenses and, in order to achieve this, surgery may be required. In some cases of severe astigmatism, spectacles or contact lenses will not be sufficient to achieve good vision and surgery will be necessary.

Corneal astigmatism can be improved by certain surgical procedures, such as refractive laser surgery and incisional corneal surgery. However, some patients have an aversion to undergoing surgery (unless essential) and any surgical procedure does involve some risks. Furthermore, the improvement in astigmatism may be unpredictable with these procedures and unsatisfactory outcomes may occur.

If a patient with corneal astigmatism also has cataracts, and cataract surgery is required, this is generally an opportunity for the patient to have both problems rectified with a single surgical procedure. Cataract surgery involves replacing the cloudy lens of the eye with an intraocular lens (IOL). The surgical technique most commonly used for cataract removal is called Phacoemulsification Surgery or Small Incision Cataract Surgery. The procedure involves the removal of the cataract and the implantation of an IOL through a micro incision (less than 3 mm wide).

An IOL is an artificial lens, usually formed of acrylic, silicone or PMMA (polymethylmethacrylate), which has a similar shape to a natural lens. An IOL is designed to reside inside the eye and focus light on the retina. In modem cataract surgery, the chosen IOL inserted has a refracting power (dioptre), that will enable the patient to see clearly in the distance without spectacle correction. Spectacles are usually required for close vision. Some patients having cataract surgery, prefer to see clearly at close range without spectacles, and an IOL is inserted with an appropriate refracting power to achieve this outcome. These patients often require spectacles for distance vision. Other patients prefer to not wear spectacles for either distance vision or near vision, and multifocal IOLs can be inserted to achieve this. However, clear vision without spectacles for distance, near or intermediate vision, is not possible if a patient has significant corneal astigmatism, unless an IOL is chosen that also corrects for astigmatism (a toric IOL).

Many cataract patients also have astigmatism that can now be corrected by surgery. Where a cataract patient also has astigmatism, the astigmatism and the cataract can both be remedied during a single surgical procedure. There are several approaches to correcting astigmatism at the time of cataract surgery, including: incision placement on the steep axis of the cornea, single or paired peripheral corneal relaxing incisions (PCRI's) and toric IOL implantation.

The methods of incision placement on the steep axis and PCRI's have a number of undesirable complications, such as unpredictability of effect, insufficient effect, infection risk and more difficult surgery. Largely because of the unpredictability of effect, these procedures (if required) are usually performed as a subsequent procedure, at a later date to the cataract surgery. This requires a second episode of treatment which involves extra costs to the patient and to any health insurance providers. Another common criticism of these procedures is that the treating nomograms are complex and confusing. The incisions have to be adapted for age, location, and degree of astigmatism. As a result of such limitations, these methods are not generally preferred in the surgical treatment of corneal astigmatism in patients who also have cataracts.

Another method to correct residual astigmatism after cataract surgery is laser assisted in-situ keratomileusis (LASIK). This method cannot be done at the time of cataract surgery. It is useful as a technique but the excimer laser treatment is expensive, involves a second episode of care and requires specialised expertise to obtain optimum results. Many cataract surgeons do not perform excimer laser treatment routinely in their practices. Other alternative procedures to correct post operative refractive errors include an IOL exchange (which involves a risk of complications and is best avoided), and a secondary supplementary toric IOL placed in the eye in the sulcus (in front of the existing IOL), this supplementary IOL is expensive.

Due to the above problems with alternative procedures, toric IOL implantation has become a preferred option for correcting corneal astigmatism in patients requiring cataract surgery. The toric IOL implantation procedure requires only minor adjustments to normal cataract surgical techniques. It generally provides favourable results in terms of unaided vision and involves relatively low risks. Also, the desirability of this procedure has recently been further enhanced by the availability of an increasing range of high quality toric IOL's. It is estimated that up to 50 percent of cataract patients have astigmatism that can be corrected by the implantation of a toric IOL.

In preparation for the implanting of a toric IOL in a patient's eye, careful measurements are made preoperatively to determine the patient's axis of corneal astigmatism (being the steep axis) and the magnitude of the astigmatism to be corrected. The angle of this axis and the magnitude of the astigmatism are recorded. The magnitude of the astigmatism will determine the type (i.e. toric power) of toric IOL which is to be implanted. The angle of the axis will be used to align the toric IOL in the correct orientation so as to overcome or ameliorate the effect of the astigmatism.

Prior to commencing surgery, and before any periocular injectable anaesthetic or general anaesthetic is given to the patient, the eye to be operated on is generally marked in the following manner. With the patient sitting upright and looking straight ahead, the limbus (which is the circular junction zone of the cornea and the sclera) at 0°, 90° and 180° (usually) is marked with a skin marking pen. These marks act as the reference points during surgery.

These marks are used to indicate the horizontal and vertical reference meridians. It is imperative to identify these meridians because they will be used to further identify the desired meridians for the incision and IOL alignment. The reference marks should be identified with the patient in an upright position as the eye typically rotates when the patient is supine. The 0 degree horizontal reference plane is on the patient's left side of each eye, at the 3 o'clock position. The 180 degree horizontal reference plane is on the patient's right side of each eye, at the 9 o'clock position. The inferior limbus is the 90 degree vertical reference plane and is at the 6 o'clock position.

Pre-operative marking of a patient's eye, for indicating the horizontal and/or vertical meridians of the eye, is typically required in several types of eye surgery involving IOL's, including cataract surgery with a toric IOL, repositioning a toric IOL (if misaligned) and inserting a supplementary toric IOL or a phakic IOL. Similar eye marking is also required in astigmatic corneal refractive laser procedures.

There are a number of methods presently in use for placing reference marks on the cornea of a patient's eye. Many surgeons use a free hand method with a surgical pen to mark the horizontal and vertical meridians. This method relies on the surgeon placing the marks at the limbus at 0°, 180° and 270° (or at 0°, 180° and 90°). Another method involves the use of a reference marker tool having marking elements oriented in a way which enable marks to be placed on the cornea in the desired positions. These marking elements are able to be inked, for instance by an appropriate ink pad or by using a marking pen, prior to being pressed against the cornea for making the desired marks.

Several reference marker instruments have been developed for pre-operative use in toric eye surgery. Known toric reference marker instruments include the Cionni™ toric reference marker (9-840), the ASICO™ toric reference marker (AE-2793S), the Ambler™ toric reference marker (9-840) and the Whitehouse toric reference marker. These toric reference marker instruments are typically formed of stainless steel and as a single unit comprising an elongated handle and, at one end, a semi-circular frame having a desired number of marking elements extending forwardly from the frame. The marking elements are generally shaped as pointed ridges which can be inked with a marking pen (or ink pad) and then pressed onto the cornea of the patient's eye. The fixed elongated handle is typically positioned in a plane which is substantially perpendicular to the plane of the frame so that, when using the marker instrument, the surgeon can apply a ‘direct’ approach (meaning that the tool and the surgeon's hand approach the eye from directly in front of the eye). Other stainless steel markers exist with the handle horizontal to the frame, such as the ASICO™ AE-2793D.

Prior to marking the patient's eye, a desired amount (typically one drop) of topical anaesthetic is applied to the eye. The pointed ridges of the reference marker instrument are then coated with a marking pen or pad (having sterilized ink). With the patient looking straight ahead, the reference marker instrument is held so that the pointed ridges align with the relevant meridians of the eye. Typically, for toric IOL implantation surgery, the reference marker instrument will have three marking elements, located at 0°, 180° and 90°. The pointed ridges of the two marking elements located at 0° and 180° are aligned with the patient's 0° and 180° meridians. The marker instrument is then moved forward towards the eye, under direct view of the surgeon, so that the three pointed ridges touch the limbus at 0°, 180° and 90°. Alternatively, many surgeons like to use a slit lamp when marking the eye (which is like a vertically mounted microscope that enables the surgeon to view a patient's eye under magnification.) Sometimes a surgeon may choose to mark the steep axis by making a small nick in the limbal blood vessels to (cause bleeding).

The patient is then prepared and made ready for surgery.

If desired, marking the incisional and desired axis of IOL alignment can subsequently be accomplished by using an appropriate toric axis marker, such as the Cionni™ toric axis marker (9-841). Such an axis marker can be utilised so as to mark the eye at the desired meridian(s) for the incision or IOL axis.

During toric IOL implantation surgery, a toric IOL is inserted inside the capsule of the lens, after the cataract has been removed. The Toric IOL is then rotated to the correct axis of orientation. The toric IOL typically has markings at or near both haptic/optic junctions. These marking indentations are aligned exactly with the steep corneal astigmatic axis, which can be in any axis from 0° to 180°. A particularly suitable toric IOL for use in the above procedure is the AcroSof™ toric IOL. This toric IOL is an acrylic polymer that has ultraviolet and blue light filters and which has been FDA approved in the USA. Other toric IOLs have become available such the Centreflex™ Toric IOL.

The abovementioned stainless steel marker instruments are re-usable but they have a number of limitations in design and use. One limitation is that these instruments are somewhat cumbersome to use. The fixed, elongated handle means that the surgeon is limited in relation to his or her position relative to the patient and this may require the surgeon to stand (or sit) in a position which is not the preferred or most comfortable position. The surgeon holds the instrument in his or her dominant hand, and must approach the eye of the patient, in the manner required by the fixed arm, either bringing it from the side, or straight ahead. The Whitehouse TM marker has a freely mobile frame connected to a weighted ball, housed in a stainless steel sleeve. This marker frequently causes mis-alignment of the axes, if the marker frame catches the lashes and rotates. This marker cannot be used at the slit lamp because of the limited working distance available. Another problem with these markers is that they mark too peripherally onto the limbus, rather than on to the cornea. A further problem with all stainless steel markers is that they obscure the anatomy of the limbus, as one approaches the eye to be marked.

One common problem with the abovementioned stainless steel instruments is that they generally have a thin neck portion, which connects the semi-circular frame to the elongated handle. This thin neck portion is relatively weak and can quite easily be bent or broken. Once broken, the instrument is generally useless and will need to be discarded.

Another problem is that the abovementioned stainless steel instruments are expensive and, although they can be re-used, they have to be sterilized after each use. Because of the cost, the purchase of several instruments is not always possible or desirable. The cost of purchase of these instruments may limit the use of preoperative markers, and surgeons may decide to use a toric IOL without marking the eye first, which is clearly undesirable, or may decide not to use toric IOLs altogether, which may not be in the best interests of the patient.

If the instrument has to be used several times during an operating list or if the instrument is rendered unsterile (e.g. because of mis-handling), it has to be re sterilized. This reduces efficiency in the operating theatre as time is wasted in sterilizing the instrument each time this is required.

Any reference herein to known prior art does not, unless the contrary indication appears, constitute an admission that such prior art is commonly known by those skilled in the art to which the invention relates, at the priority date of this application.

Wherever it is used, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

### SUMMARY OF THE INVENTION

According to the present invention, there is provided an eye marking device, for marking an eye of a patient prior to eye surgery, said eye marking device comprising:
a frame comprising a front face, a rear face and at least one marking tab extending from the front face;
a removably attachable handle; and
a handle connection means adapted to enable removable attachment of the handle to the frame in more than one orientation.

This versatility enables surgical comfort, and the ability to use at the slit lamp by the surgeon, if required.

Preferably, the handle connection means is adapted to enable the handle to be attached to the frame so that it extends selectively from a first side or a second side of the frame. In one preferred embodiment, the handle connection means generally comprises at least one protrusion on the frame and a corresponding hole in the handle for receiving said protrusion. The protrusion preferably extends from the rear face of the frame. In an alternative embodiment, the handle connection means comprises at least one hole extending from a surface of the frame and a corresponding protrusion in the handle adapted to be inserted into the hole. In this embodiment, the hole is preferably located in the rear face of the frame.

As will be appreciated, the handle connection means may include a number of alternative forms. For instance, in alternative embodiments of the invention, the frame may include holes, or perhaps a channel, located in a side edge of the frame adapted to receive one end of the handle (or a protrusion extending from one end of the handle). In another alternative embodiment, the frame may have a number of protrusions extending from the side edge of the frame, each of which is adapted to be received in a complementary shaped hole in the handle.

The handle will typically include an attachment end and a free end (although it is possible for the handle to have attachment elements at both ends). In the embodiment in which the handle includes a hole for receiving a protrusion on the rear face of the frame, the hole is typically located adjacent the attachment end of the handle. In the alternative embodiment in which the handle includes a protrusion adapted to be inserted into a hole in the rear face of the frame, the protrusion is typically located adjacent the attachment end of the handle.

In a particularly preferred embodiment, the frame comprises a substantially semi-circular shape having a first end portion, a second end portion and a base portion intermediate said first and second end portions. Preferably, the frame comprises three marking tabs extending from the front face, said marking tabs being located adjacent the first end portion, the second end portion and the base portion.

The handle connection means preferably comprises a first protrusion located between the first end portion and the base portion of the frame and a second protrusion located between the second end portion and the base portion, each of said first and second protrusions being adapted to engage with the corresponding hole in the handle.

It is further preferred that the first protrusion and the corresponding hole are adapted to enable the handle, when connected to the frame, to extend from the frame in, selectively, a substantially horizontal direction or a direction which is about 45° to the horizontal.

It is still further preferred that the second protrusion and the corresponding hole are adapted to enable the handle, when connected to the frame, to extend from the frame in, selectively, a substantially horizontal direction or a direction which is about 45° to the horizontal.

The handle is preferably between about 50 mm and 90 mm in length. A particularly preferred length is about 60 mm.

In a further preferred embodiment of the invention, the frame also comprises a tail handle adjacent to the base portion extending from the rear face of the frame. This tail handle is typically integrally formed with the frame. The tail handle is adapted to be gripped between the thumb and a finger of the surgeon. The length of this tail handle is typically between about 7 mm and 15 mm. A preferred length is about 10 mm. This tail handle enables the device to be used without the removably attachable handle.

The rear face of the frame preferably also includes three alignment markings corresponding with the three marking tabs which extend from the front face. The three marking tabs will typically have shaped ends and each end is preferably contoured to correspond with the contour of the eye. These marking tabs are typically oriented to one another at 0°, 90° and 180°.

The shaped ends of the marking tabs are preferably adapted to be inked and pressed against the eye of the patient in order to form 3 alignment marks about the cornea of the eye at 0°, 90° and 180° positions.

In one preferred embodiment, the inking of the shaped ends of the marking tabs is done as part of the manufacturing process and the shaped ends are covered by an appropriate cover so as to prevent the ink from drying prior to use. Appropriate covers may include caps over the shaped ends or a plastic cover sheet.

In an alternative embodiment, the eye marking device may be provided with one or more sterile inked pads for inking the shaped ends of the marking tabs (when required).

In a further alternative embodiment, the eye marking device may be provided together with inking means, such as one or more inked pads, in an arrangement wherein the shaped ends are in contact with the inked pads until the eye marking device is required for use by the surgeon.

Preferably, the above described eye marking device will be formed of plastic. A particular benefit of plastic is that it makes the device cheap to manufacture thereby enabling the device to be disposable and a new (sterile) device can be used for each surgical procedure. This then avoids the requirement for sterilizing the device in between such procedures and thereby avoids the significant down-time involved with such sterilization. The device may, preferably be formed of a clear plastic polymer, to assist visualization of the anatomical limbus during marking.

The abovementioned ability to have several possible configurations for the removably attachable handle is a particularly beneficial feature of the above described device as it gives the surgeon a choice of options in terms of where he or she can be positioned relative to the patient. This enhances the convenience of the surgeon and the accuracy in using the marker instrument.

### BRIEF DESCRIPTION OF THE DRAWINGS

An embodiment or embodiments of the present invention will now be described, by way of example only, with reference to the accompanying drawings, in which:

FIG. 1 is a top, rear perspective view of an eye marking device (excluding the removably attachable handle) according to a preferred embodiment of the subject invention.

FIG. 2 is a rear plan view of the eye marking device shown in FIG. 1.

FIG. 3 is a bottom front perspective view of the eye marking device shown in FIG. 1.

FIG. 4 is a front plan view of the eye marking device shown in FIG. 1.

FIG. 5 is a top view of the eye marking device shown in FIG. 1.

FIG. 6 is a bottom view of the eye marking device shown in FIG. 1.

FIG. 7 is a side view of the eye marking device shown in FIG. 1.

FIG. 8 is an exploded top, rear perspective view of the eye marking device shown in FIG. 1 together with the removably attachable handle.

FIG. 9 is a top rear perspective view of the eye marking device shown in FIG. 1 with the removably attachable handle attached thereto.

FIG. 10 is a rear plan view of the eye marking device and removably attachable handle shown in FIG. 9.

FIG. 11 is a top view of the eye marking device and removably attachable handle shown in FIG. 9.

FIG. 12 is a rear view (or front view) of the removably attachable handle according to a preferred embodiment of this invention.

FIG. 13 is a top view of the removably attachable handle shown in FIG. 12.

FIG. 14 is a top rear view (or top front view) of the removably attachable handle shown in FIG. 12.

FIG. 15 is an exploded view of the eye marking device and removably attachable handle of the present invention illustrating different optional orientations in which the handle can be attached to the frame of the eye marking device.

FIG. 16 is a top rear perspective view of the eye marking device shown in FIG. 1 positioned against an eye globe.

FIG. 17 is a top rear perspective view of the eye marking device shown in FIG. 9 (with handle) pressed against an eye globe.

### DETAILED DESCRIPTION OF A PREFERRED EMBODIMENT

A preferred embodiment of the present invention is described below with reference to the above drawings. As shown in FIGS. 8 and 9, a preferred embodiment of the eye marking device of the present invention includes a frame 1 and a removably attachable handle 2.

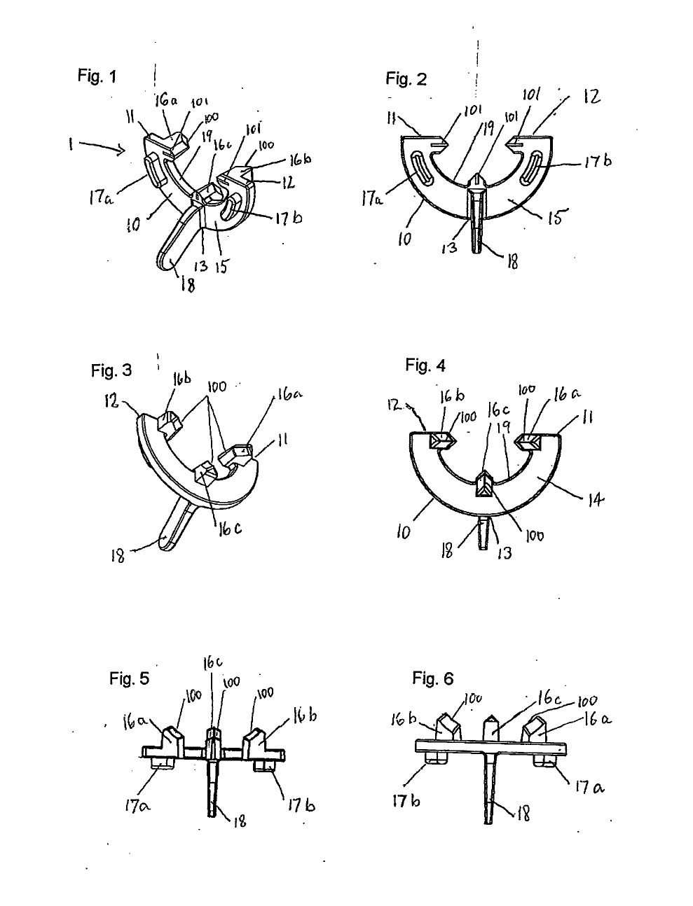

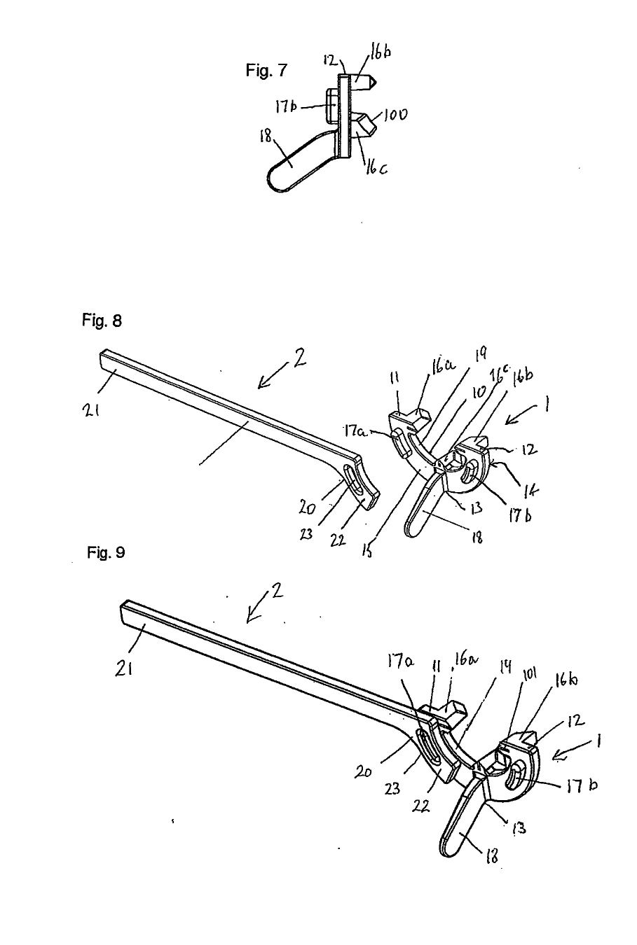

As more particularly shown in FIGS. 1 to 7, the frame 1 has a substantially semi-circular body 10 having a first end portion 11, a second end portion 12 and a base portion 13. The circular body 10 has a curved inner edge 19.

The frame 1 also includes a front face 14 and a rear face 15. The frame 1 further includes three marking tabs 16a, 16b and 16c which extend forwardly from the front face 14. One marking tab 16a is located adjacent the first end portion 11 of the frame 1. Another marking tab 16b is located adjacent the second end portion 12 of the frame 1. The third marking tab 16c is located adjacent the base portion 13 of the frame 1.

The frame 1 also includes two connecting protrusions 17a, 17b which extend rearwardly from the rear face 15 of the frame 1. The connecting protrusion 17a is located between the first end portion 11 and the base portion 13, although it is nearer to the first end portion 11. The connecting protrusion 17b is located between the second end portion 12 and the base portion 13, although it is nearer to the second end portion 12. In the embodiment of the invention shown in the drawings, the connecting protrusions 17a, 17b are arcuate, having a curvature which substantially corresponds with the curvature of the semi-circular body 10. It will be understood however that the arcuate shape of the connecting protrusions is not essential and other suitable shapes may be adopted.

The frame 1 further includes a tail handle 18 extending rearwardly and downwardly from the rear face 15. This tail handle 18 is dimensioned so as to be able to be conveniently clasped between the thumb and a finger of the surgeon. The downward extension of the tail handle 18 is so as to avoid the surgeon's fingers obstructing his or her vision when using the device to apply markings to a patient's eye.

Each of the three marking tabs 16a, 16b and 16c which extend forwardly from the front face 14 has a shaped end 100. Each shaped end 100 is shaped to have a contour which substantially corresponds with the contour of the cornea of a typical eye.

The rear face of the frame further includes three alignment marks 101. Each alignment mark 101 is positioned adjacent the curved inner edge 19 and directly behind each of the marking tabs 16a, 16b and 16c. The alignment marks 101 should be (as shown) in direct alignment with the shaped ends 100 so as to enable the surgeon to determine accurately where the shaped ends 100 will mark the patient's eye.

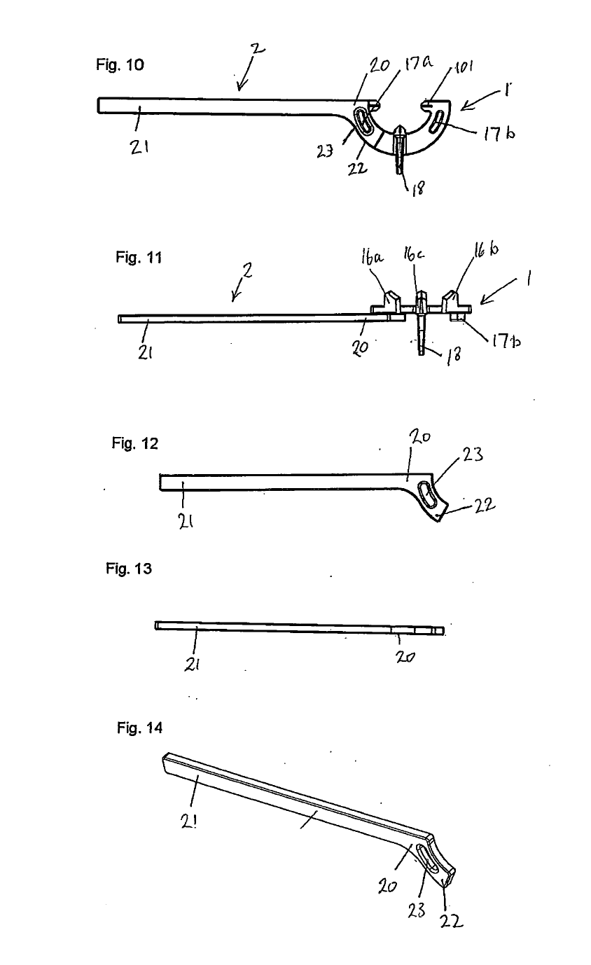

As more particularly shown in FIGS. 8 to 14, the removably attachable handle 2 generally includes an attachment end 20 and a free end 21. The attachment end comprises a curved extension 22 and a hole 23. The hole 23 is of complementary shape to that of the connecting protrusions 17a, 17b. In the embodiment shown, the hole 23 therefore has an arcuate shape corresponding with the arcuate shape of the connecting protrusions 17a, 17b. The shape of the curved extension 22 clearly accommodates the curved shape of the hole 23. As will be appreciated, the hole could be of any shape provided that it is complementary with the shape of the corresponding connecting protrusions and, consequently, the shape or design of the attachment end of the handle may vary accordingly.

In the embodiment of the invention shown in FIGS. 8 to 14, the curved extension 22 has an inner curved edge and an outer curved edge which correspond with the curved inner edge 19 and the curved outer edge of the semi-circular body 10, thereby creating a neat appearance when joined to the frame 1 and avoiding any vision obstruction when the device is in use.

As is more clearly shown in FIGS. 8, 9, 10, 15 and 17, the handle 2 is connected to the frame 1 by inserting one of the connecting protrusions 17a, 17b (on the rear face 15) into the hole 23 (in the attachment end 20 of the handle 2).

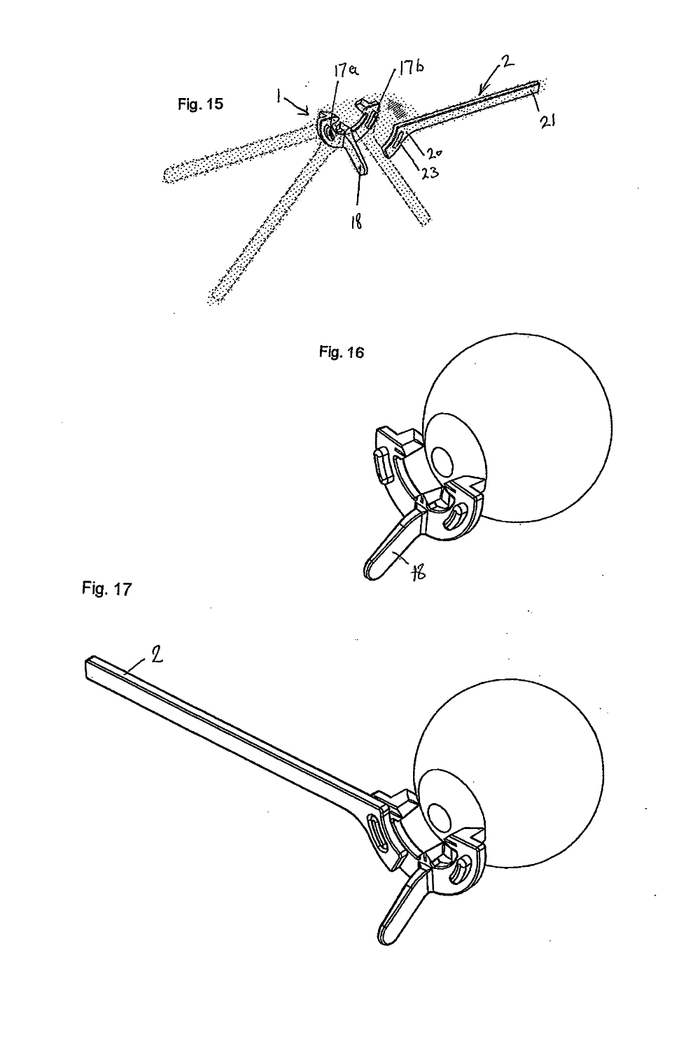

As is best illustrated in FIG. 15, in the embodiment of the invention shown in the drawings, the handle 2 can be attached to the frame 1 in four different orientations, being: horizontal to the left; about 45° to the horizontal (to the left); horizontal to the right; and about 45° to the horizontal (to the right). This is a particularly preferred feature of the preferred embodiment of the invention as it enhances the useability and versatility of the eye marker device.

As shown in FIG. 17, the eye marking device of this invention can be used to mark the eye of a patient. The surgeon will typically hold the handle 2 and move the device towards the patient's eye until the shaped ends 100 of the marking tabs 16a, 16b and 16c touch the eye. The shaped ends 100, which are pre-loaded with ink, are then able to mark the eye in the three places corresponding to the location of the shaped ends 100. These marks are typically formed about the circumference of the cornea (at the limbus) of the patient's eye.

As more particularly shown in FIG. 16, the eye marking device of the present invention can also be used without the removably attachable handle 2. When the handle 2 is not attached, the tail handle 18 is able to be grasped by the surgeon and the shaped ends 100 can be pressed against the patient's eye thereby marking the eye as explained above.

As will be appreciated by the above description of a preferred embodiment, the incorporation of a removably attachable handle in an eye marking device is particularly beneficial in that it enables a surgeon to have numerous options in terms of how he or she holds the eye marking device and the position which he or she adopts when using the marking device. This versatility substantially enhances the comfort and ease of use of the surgeon when marking the eye of a patient.

As will be appreciated, the subject invention is not necessarily restricted to devices for marking an eye prior to eye surgery and the subject invention may have use in relation to other surgical tools.

It will be understood that the invention disclosed and defined herein extends to all alternative combinations of two or more of the individual features mentioned or evident from the text. All of these different combinations constitute various alternative aspects of the invention.

While particular embodiments of this invention have been described, it will be evident to those skilled in the art that the present invention may be embodied in other specific forms without departing from the essential characteristics thereof.

The present embodiments described herein (including the drawings) are therefore to be considered in all respects as illustrative, and not restrictive, of the present invention, and all modifications which would be obvious to those skilled in the art are therefore intended to be embraced therein.

1. An eye marking device, for marking an eye of a patient prior to eye surgery, said eye marking device comprising:
a frame comprising a front face, a rear face and at least one marking tab extending from the front face;
a removably attachable handle; and
handle connection means adapted to enable removable attachment of the handle to the frame in more than one orientation.

2. An eye marking device according to claim 1, wherein the handle connection means is adapted to enable the handle to be attached to the frame so that it extends selectively from a first side or a second side of the frame.

3. An eye marking device according to claim 1, wherein the handle connection means comprises at least one protrusion on the rear face of the frame and a corresponding hole in the handle for receiving said protrusion.

4. An eye marking device according to claim 1, wherein the handle connection means comprises at least one hole extending from the rear face of the frame and a corresponding protrusion in the handle adapted to be inserted into the hole.

5. An eye marking device according to claim 1, wherein the handle comprises an attachment end and a free end.

6. An eye marking device according to claim 3, wherein the hole in the handle is located adjacent the attachment end.

7. An eye marking device according to claim 4, wherein the protrusion on the handle is located adjacent the attachment end.

8. An eye marking device according claim 1, wherein the frame comprises a substantially semi-circular shape having a first end portion, a second end portion and a base portion intermediate said first and second end portions.

9. An eye marking device according to claim 8, wherein the frame comprises three marking tabs extending from the front face, said marking tabs being located adjacent the first end portion, the second end portion and the base portion.

10. An eye marking device according to claim 3, wherein the handle connection means comprises a first protrusion located between the first end portion and the base portion of the frame and a second protrusion located between the second end portion and the base portion, each of said first and second protrusions being adapted to engage with the corresponding hole in the handle.

11. An eye marking device according to claim 10 wherein the first protrusion and the corresponding hole are adapted to enable the handle, when connected to the frame, to extend from the frame in, selectively, a substantially horizontally direction or a direction which is about 45° to the horizontal.

12. An eye marking device according to claim 10 wherein the second protrusion and the corresponding hole are adapted to enable the handle, when connected to the frame, to extend from the frame in, selectively, a substantially horizontally direction or a direction which is about 45° to the horizontal.

13. An eye marking device according to claim 8, wherein the frame further comprises a tail handle adjacent the base portion extending from the rear face of the frame.

14. An eye marking device according to claim 13, wherein the tail handle is integrally formed with the frame.

15. An eye marking device according to claim 1, wherein the rear face of the frame comprises three alignment markings corresponding with the three marking tabs which extend from the front face.

16. An eye marking device according to claim 9, wherein the three marking tabs have shaped ends, each end being contoured to correspond with the contour of the eye.

17. An eye marking device according to claim 1, wherein the marking tabs are oriented to one another at 0°, 90° and 180°.

18. An eye marking device according to claim 16, wherein the shaped ends of the marking tabs are adapted to be inked and pressed against the eye of the patient in order to form 3 alignment marks about the cornea of the eye at 0°, 90° and 180° positions.

19. An eye marking device according to claim 1, wherein the device is formed of plastic.

20. An eye marking device for marking an eye of a patient prior to eye surgery, said eye marking device being substantially as hereinbefore described with reference to any one of the drawings.
